(19) United States
(12) Patent Application Publication
(10) Pub. No.: US 2003/0073890 A1
(43) Pub. Date: Apr. 17, 2003

(54) Plethysmographic signal processing method and system

(76) Inventor: D. Alan Hanna, Boulder, CO (US)

Correspondence Address: Marsh Fischmann & Breyfogle LLP, 3151 S. Vaughn Way, Suite 411, Aurora, CO, 80014, US

(21) Appl. No.: 09/975,289

(22) Filed: Oct. 10, 2001

(51) Int. Cl. A61B 5/00

(52) U.S. Cl. 600/323

(57) ABSTRACT

The present invention provides a plethysmographic signal processing method and system that achieves improved S/N ratios leading to improved patient heart rate estimates and improved plethysmographic waveform displays. The plethysmographic signal processing method and system of the present invention may be implemented using analog and/or digital components within a pulse oximeter. In one embodiment, first and second plethysmographic signals S₁, S₂ associated with first and second wavelengths, respectively (e.g., infrared and red), are received on first and second channels 210, 212. First and second multipliers 214, 216 multiply the first and second plethysmographic signals S₁, S₂ by first and second multiplication factors T₁, T₂. A summer 218 sums the products from the first and second multipliers 214, 216 to output a composite plethysmographic signal C on an output channel 220. The composite plethysmographic signal C may then be displayed and/or utilized to make heart rate determinations and the like.

### FIELD OF THE INVENTION

The present invention relates generally to the non-invasive determination of patient heart rates from plethysmographic signals, and more particularly to achieving improved signal-to-noise ratios in plethysmographic signals used to estimate patient heart rates and the like.

### BACKGROUND OF THE INVENTION

In photoplethysmography, light signals corresponding with two or more different center wavelengths are utilized to non-invasively determine various blood analyte concentrations in a patient's blood and to obtain information regarding the patient's heart rate and the like. By way of primary example, blood oxygen saturation (SpO₂) levels of a patient's arterial blood are monitored in pulse oximeters by measuring the absorption of oxyhemoglobin (O2Hb) and reduced hemoglobin (RHb) using red and infrared light signals. The measured absorption data allows for the calculation of the relative concentrations of O2Hb and RHb, and therefore SPO₂ levels, since RHb absorbs more light than O2Hb in the red band and O2Hb absorbs more light than RHb in the infrared band, and since the absorption relationship of the two analytes in the red and infrared bands is known.

To obtain absorption data, pulse oximeters typically comprise a probe that is releaseably attached to a patient tissue site (e.g., finger, ear lobe, nasal septum, foot). The probe directs red and infrared light signals through the patient tissue site. The light signals are provided by one or more light signal sources (e.g., light emitting diodes or laser diodes) which are typically disposed in the probe. A portion of the red and infrared light signals is absorbed in the patient tissue site and the intensity of the transmitted light signals (light exiting the patient tissue site is referred to as transmitted) is detected by a detector that may also be located in the probe. The detector outputs a signal which includes information indicative of the intensities of the transmitted red and infrared light signals. The output signal from the detector may be processed to obtain separate signals associated with the red and infrared transmitted light signals (i.e., separate red and infrared plethysmographic signals or waveforms).

As will be appreciated, pulse oximeters rely on the time-varying absorption of light in the patient tissue site as it is supplied with pulsating arterial blood. The patient tissue site may contain a number of non-pulsatile light absorbers, including capillary and venous blood, as well as muscle, connective tissue and bone. Consequently, the red and infrared plethysmographic signals typically contain a large non-pulsatile, or DC, component, and a relatively small pulsatile, or AC, component. Patient heart rate can be determined by examining the time period between successive peaks in the small pulsatile AC component of the red or infrared plethysmographic signals. The small pulsatile AC component of the red or infrared plethysmographic signals can also be displayed on the monitor unit for further observation by persons involved in the treatment of the patient.

As noted, the pulsatile AC component of a pulse oximeter detector output signal is relatively small compared to the non-pulsatile DC component. Consequently, the accuracy of the heart rate determination and the information which can be obtained through visual perception of the plethysmographic signals on a display can be severely impacted by small amounts of noise. Noise may be introduced by factors such as, for example, motion of the patient tissue site, corruption of the transmitted light signals by ambient light, and noise inherent in the electronic and opto-electronic components of the pulse oximeter. Furthermore, in patients having high SpO₂ levels, the infrared plethysmographic signal typically has a better signal-to-noise (SIN) ratio and is preferred for visual display and heart rate determinations. However, in patients with low SpO₂ levels, the red plethysmographic signal typically has a better SIN ratio and is therefore preferred for visual display and heart rate determinations.

### SUMMARY OF THE INVENTION

Accordingly, the present invention provides a plethysmographic signal processing method and system that achieves improved S/N ratios leading to improved patient heart rate estimates and improved plethysmographic waveform displays. The plethysmographic signal processing method and system generates a composite plethysmograhic signal from two or more plethysmographic signals (e.g., red and infrared). The composite plethysmographic signal has an improved S/N ratio over the full range of patient SpO₂ levels as compared to any of the separate plethysmographic signals from which it is generated.

According to one aspect of the present invention, a plethysmographic signal processing method includes the step of receiving at least two plethysmographic signals. Each plethysmographic signal received is associated with a particular wavelength. In this regard, where there are two plethysmographic signals (e.g., in pulse oximetry), a first one of the plethysmographic signals may be associated with infrared wavelengths (e.g., wavelengths from about 800 nm to about 950 nm), and a second one of the plethysmographic signals may be associated with red wavelengths (e.g., wavelengths from about 600 nm to 700 nm). Each plethysmographic signal received is multiplied by an associated scalar multiplication factor. A composite plethysmographic signal comprising a linear combination of the plethysmographic signals is then generated by adding the results of the multiplications. The plethysmographic signals may be analog signals or digital signals. Where the plethysmographic signals are digital signals, the multiplications and additions are performed for each temporally corresponding signal sample value (i.e., each corresponding-in-time sample instance).

In the plethysmographic signal processing method, the multiplication factors may be specifically chosen to provide an improved S/N ratio for the composite signal that is generated as compared to the S/N ratios of the separate plethysmographic signals that are received over a specified range of patient SpO₂ levels (e.g., from about 40% to about 100%). In this regard, the multiplication factors may be chosen to depend upon a ratio (e.g., an R value) wherein the ratio varies in accordance with the SpO₂ level in arterial blood circulated through a patient tissue site. By way of example, where there are first and second plethysmographic signals associated with an infrared wavelength and a red wavelength, respectively, first and second multiplication factors designated T₁ and T₂ and associated with the first and second plethysmographic signals, respectively, may be specified in accordance with the following equations:

$${T}_{1} = \frac{1}{\sqrt{1 + {R}^{2}}}$$

$${T}_{2} = - \frac{R}{\sqrt{1 + {R}^{2}}}$$

In the above equations, R may be the ratio of a first differential absorption value dA₁ obtained from the first plethysmographic signal and a second differential absorption value dA₂ obtained from the second plethysmographic signal calculated as follows:

$$R = \frac{ⅆ {A}_{2}}{ⅆ {A}_{1}}$$

Multiplication factors which depend upon the R value as described above may be obtained in a number of manners. For example, prior to multiplying the plethysmographic signals by their associated multiplication factors, the R value may be computed each time it is needed using the latest differential absorption values available (e.g., from another method or system utilized in a pulse oximeter) and the multiplication factors may be then be computed using the updated R value. As may be appreciated, this is fairly computationally intensive since computation of each multiplication factor requires a multiplication, addition, square root and division operation. As an alternative, the multiplication factors may be obtained from a look-up table. The look-up table includes sets of multiplication factors that are cross-referenced with corresponding incremental R values. The look-up table may, for example, include multiplication factors corresponding with incremental R values ranging from 40% to 100%. In this regard, the R values in the look-up table may, for example, be incremented in equal increments, with the increments being between about 0.001 and about 0.1 in size.

According to another aspect of the present invention, a signal processing method for use in plethysmography includes the step of receiving first and second plethysmographic signals S₁ and S₂. The first and second plethysmographic signals S₁ and S₂ are associated with first and second wavelengths, respectively (e.g., infrared and red). A complex signal vector S=S₁+iS₂ is formed by treating the first plethysmographic signal S₁ as the real component of the complex signal vector S and treating the second plethysmographic signal S₂ as the imaginary component of the complex signal vector S. A complex transformation vector T is also formed from first and second scalar multiplication factors T₁ and T₂. In this regard, the first scalar multiplication factor T₁ is treated as the real component of the complex transformation vector T and the second scalar multiplication factor T₂ is treated as the imaginary component of the complex transformation vector T (i.e. T=T₁+iT₂). The first and second scalar multiplication factors T₁ and T₂ may depend upon an R value comprising the ratio of a differential absorption value dA₂ obtained from the second plethysmographic signal S₂ to a differential absorption value dA₁ obtained from the first plethysmographic signal S₁. The complex signal vector S is then multiplied by the complex transformation vector T to generate a composite plethysmographic signal C. The composite plethysmographic signal C achieved has an improved signal strength as compared with either of the first and second plethysmographic signals S₁ and S₂.

According to a further aspect of the present invention, a plethysmographic signal processing system includes first and second input channels for receiving first and second plethysmographic signals thereon. The first and second plethysmographic signals are associated with first and second wavelengths, respectively (e.g., infrared and red). The system also includes first and second multipliers. The first multiplier is operable to receive the first plethysmographic signal and a first scalar multiplication factor as inputs and output a first product comprising the first plethysmographic signal multiplied by the first scalar multiplication factor. The second multiplier is operable to receive the second plethysmographic signal and a second scalar multiplication factor as inputs and output a second product comprising the second plethysmographic signal multiplied by the second scalar multiplication factor. The system also includes a summer. The summer is operable to receive the first and second products as inputs and add the first and second products to output a composite signal comprising the sum of the first and second products.

The first and second plethysmographic signals may comprise continuous time signals, in which case the system of the present invention may be implemented for processing the first and second plethysmographic signals in a continuous time fashion. In the regard, the first channel, second channel, first multiplier, second multiplier, and summer may all comprise analog components. The first and second plethysmographic signals may also comprise discretized-in-time (digital) signals, in which case the system of the present invention may be implemented in software executable by a digital processor.

The first and second scalar multiplication factors may be dependent upon a ratio (e.g., an R value) that varies in accordance with an SpO₂ level in arterial blood circulated through a patient tissue site. In this regard, the ratio may be computed as follows:

$$R = \frac{ⅆ {A}_{2}}{ⅆ {A}_{1}}$$

where dA₁ and dA₂ comprise differential absorption values associated with the first and second plethysmographic signals, respectively. The first and second scalar multiplication factors, designated T₁ and T2, may be specified in accordance with the following equations:

$${T}_{1} = \frac{1}{\sqrt{1 + {R}^{2}}}$$

$${T}_{2} = - \frac{R}{\sqrt{1 + {R}^{2}}}$$

The system may compute the first and second scalar multiplication factors when needed. Alternatively, the system may further include a look-up table that has multiple pairs of pre-computed first and second scalar multiplication factors cross-referenced with corresponding incremental R values. In this regard, the pairs of first and second scalar multiplication factors may correspond with incremental R values in the range of about 40% to about 100%, with the increments being equal and between about 0.001 and about 0.1 in size.

Where it is desirable to process additional plethysmographic signals (e.g., third and fourth plethysmographic signals associated with third and fourth wavelengths), the system may include additional input channels for receiving the additional plethysmographic signals. Additional multipliers are also included. The additional multipliers are operable to receive the additional plethysmographic signals and additional scalar multiplication factors as respective inputs and output additional products comprising the respective additional plethysmographic signals multiplied by the respective additional scalar multiplication factors. The summer is then operable to receive as inputs thereto not only the first and second products, but also the additional products as well, and compute the sum of all of the products to output the composite plethysmographic signal.

These and other aspects and advantages of the present invention will be apparent upon review of the following Detailed Description when taken in conjunction with the accompanying figures.

### DESCRIPTION OF THE DRAWINGS

For a more complete understanding of the present invention and further advantages thereof, reference is now made to the following Detailed Description, taken in conjunction with the drawings, in which:

FIG. 1 is a block diagram illustrating one embodiment of an exemplary pulse oximeter within which the plethysmographic signal processing method and system of the present invention may be implemented;

FIG. 2 is a flow chart illustrating the steps of one embodiment of a plethysmographic signal processing method in accordance with the present invention;

FIGS. 3A-B are plots of exemplary complex signal vectors and complex transformation vectors formed in the steps of the plethysmographic signal processing method of FIG. 2;

FIG. 4 is a block diagram illustrating one embodiment of a plethysmographic signal processing system in accordance with the present invention;

FIG. 5 shows an exemplary look-up table having pairs of first and second multiplication factors cross-referenced with corresponding incremental R values; and

FIG. 6 is a plot of exemplary infrared plethysmographic and red plethysmographic signals and a composite signal obtained therefrom by a plethysmographic signal processing system in accordance with the present invention.

### DETAILED DESCRIPTION

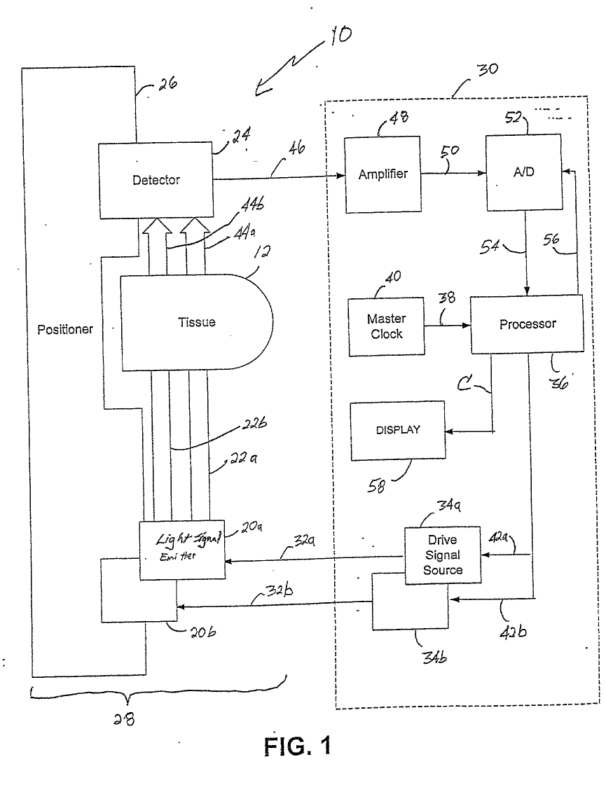

Referring to FIG. 1, there is shown an exemplary pulse oximeter 10 within which the plethysmographic signal processing method and system of the present invention may be implemented. The pulse oximeter 10 is configured for use in determining one or more blood analyte levels in a patient tissue site 12. However, the plethysmographic signal processing method and system of the present invention may be implemented in any device wherein plethysmographic signals are utilized to obtain desired information therefrom.

The pulse oximeter 10 includes two light signal emitters 20a-b (e.g., light emitting diodes or laser diodes) for emitting two light signals 22a-b centered at different predetermined center wavelengths λ₁, λ₂ through the patient tissue site 12 and on to a detector 24 (e.g., a photo-sensitive diode). The center wavelengths λ₁, λ₂ required depend upon the blood analytes to be determined. For example, in order to determine the levels of O2Hb and RHb, λ₁ may be within the infrared region of the electromagnetic spectrum (e.g., about 800-950 nm) and λ₂ may within the red region of the electromagnetic spectrum (e.g., about 600-700 nm). If more blood analyte levels are to be measured, the pulse oximeter 10 may include additional light signal emitters for emitting light signals centered at additional wavelengths.

The light signal emitters 20a-b and detector 24 may be included in a positioning device 26 to facilitate alignment of the light signals 22a-b with the detector 24. For example, the positioning device 26 may be of clip-type or flexible strip configuration adapted for selective attachment to the patient tissue site 12. The positioning device 26 may be part of a probe cable unit 28 that is connectable with a separate monitor unit 30.

The light signal emitters 20a-b are activated by a corresponding plurality of analog drive signals 32a-b to emit the light signals 22a-b. The drive signals 32a-b are supplied to the light signal emitters 20a-b by a corresponding plurality of drive signal sources 34a-b. The drive signal sources 34a-b may be connected with a digital processor 36, which is driven with a clock signal 38 from a master clock 40. The digital processor 36 may be programmed to define modulation waveforms, or drive patterns, for each of the light signal emitters 20a-b. More particularly, the digital processor 36 may provide separate digital trigger signals 42a-b to the drive signal sources 34a-b, which in turn generate the analog drive signals 32a-b. The drive signal sources 34a-b, processor 36 and clock 40 may all be housed in the monitor unit 30.

Transmitted light signals 44a-b (i.e., the portions of light signals 22a-b exiting the patient tissue site 12) are detected by the detector 24. The detector 24 detects the intensities of the transmitted signals 44a-b and outputs a current signal 46 wherein the current level is indicative of the intensities of the transmitted signals 44a-b. As may be appreciated, the current signal 46 output by the detector 24 comprises a multiplexed signal in the sense that it is a composite signal including information about the intensity of each of the transmitted signals 44a-b. Depending upon the nature of the drive signals 32a-b, the current signal 46 may, for example, be time-division multiplexed, wavelength-division multiplexed, or code-division multiplexed.

The current signal 46 is directed to an amplifier 48, which may be housed in the monitor unit 30 as is shown. The amplifier 48 converts the current signal 46 to a voltage signal 50 wherein a voltage level is indicative of the intensities of the transmitted signals 22a-b. The amplifier 48 may also be configured to filter the current signal 46 from the detector 24 to reduce noise and aliasing. By way of example, the amplifier 48 may include a bandpass filter to attenuate signal components outside of a predetermined frequency range encompassing modulation frequencies of the drive signals 32a-b.

Since the current signal 46 output by the detector 24 is a multiplexed signal, the voltage signal 50 is also a multiplexed signal, and thus, the voltage signal 50 must be demultiplexed in order to obtain signal portions corresponding with the intensities of the transmitted light signals 44a-b. In this regard, the digital processor 36 may be provided with demodulation software for demultiplexing the voltage signal 50. In order for the digital processor 36 to demodulate the voltage signal 50, it must first be converted from analog to digital. Conversion of the analog voltage signal 50 is accomplished with an analog-to-digital (A/D) converter 52, which may also be included in the monitor unit 30. The A/D converter 52 receives the analog voltage signal 50 from the amplifier 48, samples the voltage signal 50, and converts the samples into a series of digital words 54 (e.g., eight, sixteen or thirty-two bit words), wherein each digital word 54 is representative of the level of the voltage signal 50 (and hence the intensities of the transmitted light signals 44a-b) at a particular sample instance. In this regard, the A/D converter 52 should provide for sampling of the voltage signal 50 at a rate sufficient to provide for accurate tracking of the shape of the various signal portions comprising the analog voltage signal 50 being converted. For example, the A/D converter 52 may provide for a sampling frequency at least twice the frequency of the highest frequency drive signal 32a-b, and typically at an even greater sampling rate in order to more accurately represent the analog voltage signal 50.

The series of digital words 54 is provided by the A/D converter 52 to the processor 36 to be demultiplexed. More particularly, the processor 36 may periodically send an interrupt signal 56 (e.g., once per every eight, sixteen or thirty-two clock cycles) to the A/D converter 52 that causes the A/D converter 52 to transmit one digital word 54 to the processor 36. The demodulation software may then demultiplex the series of digital words 54 in accordance with an appropriate method (e.g., time, wavelength, or code) to obtain two digital signal portions indicative of the intensities of each of the transmitted light signals 44a-b.

The demultiplexed digital signal portions comprise first and second plethysmographic signals S₁ and S₂ associated with the two separate center wavelengths λ₁, λ₂ (e.g., infrared and red) of the transmitted light signals 44a-b. The first and second plethysmographic signals S₁ and S₂ may then be processed to obtain desired information therefrom such as O2Hb and RHb levels in the patient tissue site 12 as well as the patient's heart rate. In this regard, the first and second plethysmographic signals S₁ and S₂ may be processed in accordance with the steps of the plethysmographic signal processing method of the present invention in order to generate a composite plethysmographic signal C having an improved SIN ratio as compared to either of the first and second plethysmographic signals S₁ and S₂. The composite plethysmographic signal C may then be displayed on a display device 58 of the monitor unit 30 and processed further to obtain the patient's heart rate.

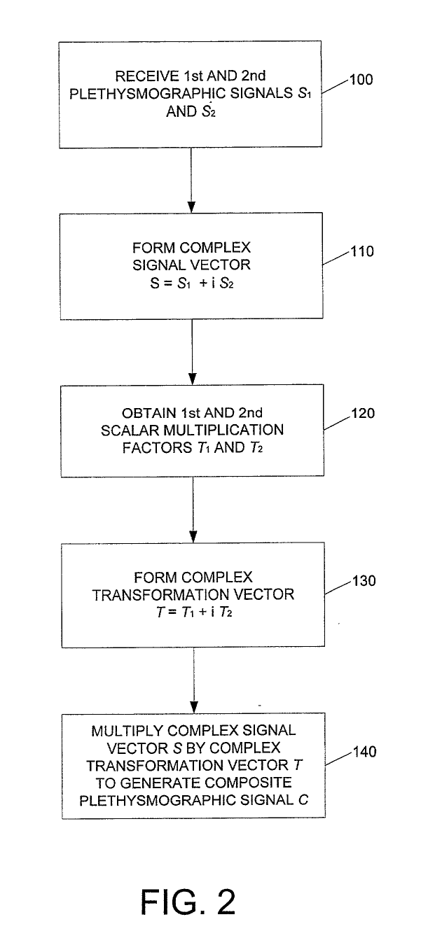

Referring now to FIG. 2 the steps of one embodiment of a plethysmographic signal processing method in accordance with the present invention are shown. The method begins with step 100 wherein first and second plethysmographic signals S₁ and S₂ are received. In this regard, the plethysmographic signals S₁ and S₂ may be received from the detector of a pulse oximeter probe, either directly or after appropriate amplification and filtering. Typically, the plethysmographic signals S₁ and S₂ will be associated with infrared and red wavelength optical signals transmitted by the probe through a patient tissue site, although plethysmographic signals associated with other wavelength optical signals may be processed in accordance with the steps of the plethysmographic signal processing method described herein.

The infrared and red plethysmographic signals S₁ and S₂ are separately processed to obtain an R value associated therewith. The R value is defined as the ratio of red optical signal absorption in the patient tissue site to infrared optical signal absorption in the patient tissue site and provides information regarding oxygen saturation of hemoglobin in arterial blood circulated through the patient tissue site (higher R values indicate lower oxygen saturation levels). In this regard, the R value may computed as the ratio of a red delta absorption value dA_{Red} to an infrared delta absorption value dA_{Infrared} (i.e. R=dA_{Red}/dA_{Infrared}). The delta absorption values dA_{Red}, dA_{Infrared} and the R value depending thereon may, for example, be obtained from the infrared and red plethysmographic signals S₁ and S₂ as described in U.S. Pat. No. 5,934,277 entitled “SYSTEM FOR PULSE OXIMETRY SPO2 DETERMINATION”, the disclosure of which is incorporated herein in its entirety.

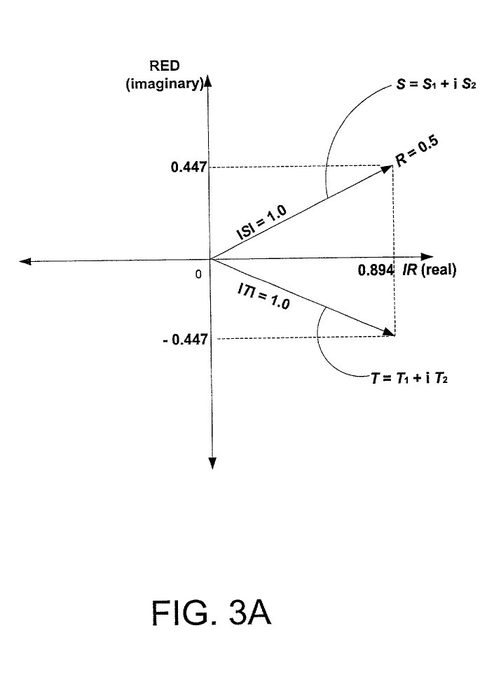

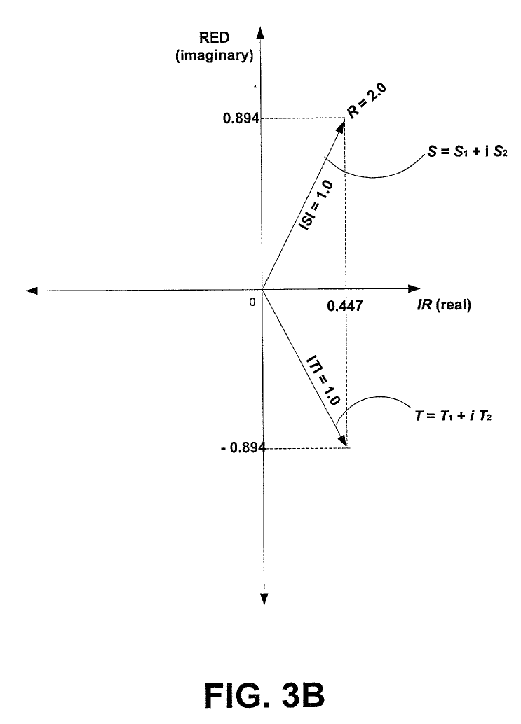

In step 110, a complex signal vector S is formed using the received plethysmographic signals S₁ and S₂. The complex signal vector S is formed by treating the first plethysmographic signal S₁ as the real component of the complex signal vector S and treating the second plethysmographic signal S₂ as the imaginary component of the complex signal vector S (i.e., S=S₁+iS₂). In this regard, exemplary complex signal vectors S formed from infrared and red plethysmographic signals S₁ and S₂ at a particular instant in time having respective R values of 0.5 (normal oxygen saturation) and 2.0 (low oxygen saturation) are illustrated in FIGS. 3A-B. In FIGS. 3A-B, the complex signal vectors S have been normalized to have magnitudes of 1.0 and plotted on a coordinate system where the infrared component of the complex signal vector S corresponds with the real axis and the red component of the complex signal vector S corresponds with the imaginary axis. The slopes of the complex signal vectors S correspond with their respective R values.

In step 120, first and second scalar multiplication factors T₁ and T₂ are obtained. The first and second scalar multiplication factors T₁ and T₂ are chosen such that multiplication of the complex signal vector S (see step 140) by a complex transformation vector T formed from the multiplication factors (see step 130) rotates the complex signal vector S onto the real axis of the coordinate system. In this regard, the first and second scalar multiplication factors T₁ and T₂ depend upon the R value and are given by the following equations:

$${T}_{1} = \frac{1}{\sqrt{1 + {R}^{2}}}$$

$${T}_{2} = - \frac{R}{\sqrt{1 + {R}^{2}}}$$

Where the first and second plethysmographic signals S₁ and S₂ are associated with optical signal wavelengths other than infrared and red, the first and second multiplication factors T₁ and T₂ may be given by different equations and depend upon factors other than the R value.

The first and second scalar multiplication factors T₁ and T₂ may be obtained in several manners. They may be computed as needed using the most recently updated R value in accordance with above equations for T₁ and T₂. Alternatively, pairs of first and second scalar multiplication factors T₁ and T₂ corresponding with various incremental R values can be computed in advance in accordance with the above equations for T₁ and T₂ and stored in a lookup table. When needed, the first and second scalar multiplication factors T₁ and T₂ corresponding with the most recently updated R value are selected from the lookup table.

In step 130, a complex transformation vector T is formed using the scalar multiplication factors T₁ and T₂ obtained in step 120. In this regard, the complex transformation vector T is formed by treating the first scalar multiplication factor T₁ as the real component of the complex transformation vector T and treating the second scalar multiplication factor T₂ as the imaginary component of the complex transformation vector T (i.e., T=T₁+iT₂). Exemplary complex transformation vectors T formed using the scalar multiplication factors T₁ and T₂ obtained in accordance with the formulas for T₁ and T₂ described in connection with step 120 using respective R values of 0.5 (normal oxygen saturation) and 2.0 (low oxygen saturation) are illustrated in FIGS. 3A-B.

In step 140, the complex signal vector S is multiplied by the complex transformation vector T to generate a composite plethysmographic signal C. Multiplication of the complex signal vector S by the complex transformation vector T results in rotation of the complex signal vector S onto the real axis of the coordinate system because appropriate scalar multiplication factors T₁ and T₂ have been employed in forming the complex transformation vector T. In this regard, as can be seen for the exemplary complex signal vectors S and complex transformation vectors T illustrated in FIGS. 3A-B, the complex transformation vectors T are the reflections of the complex signal vectors S across the real axis (i.e., they are the complex conjugates of the complex signal vectors S). Rotation of the complex signal vector S onto the real axis results in a composite plethysmographic signal C which has improved signal strength as compared with either of the first and second plethysmographic signals S₁ and S₂.

The following two examples illustrate the improvements in signal strength that are obtained by processing the red and infrared plethysmographic signals in accordance with the method of the present invention.

### EXAMPLE 1

In the following example, it is assumed that R=0.5 and that the magnitude of the complex signal vector S is 1.0. Such a situation is representative of a normal (i.e., high SpO₂ saturation) patient. As is illustrated in FIG. 3A, the slope of the complex signal vector S formed by combining the infrared and red signals S₁, S₂ has a slope of 0.5 and a length of 1.0. The projection of the complex signal vector S onto the infrared axis is 0.894 and the projection of the complex signal vector S onto the red axis is 0.447. Thus, the infrared signal S₁ has a better S/N ratio than the red signal S₂. The complex signal vector S is rotated into the real axis by multiplying the complex signal vector S by the complex signal transformation vector:

$$\begin{matrix}T = {T}_{1} + i {T}_{2} \\ = \frac{1}{\sqrt{1 + {R}^{2}}} - i \frac{R}{\sqrt{1 + {R}^{2}}} \\ = \frac{1}{\sqrt{1 + {0.5}^{2}}} - i \frac{0.5}{\sqrt{1 + {0.5}^{2}}} \\ = 0.894 - 0.447 i\end{matrix}$$

The following result is obtained:

$$\begin{matrix}S * T = ( 0.894 + 0.447 i ) ( 0.894 - 0.447 i ) \\ = 0.7992 - 0.3996 i + 0.3996 i - 0.1998 {i}^{2} \\ = 1.0\end{matrix}$$

The result obtained is nearly an 11% increase in signal strength as compared with using the infrared signal by itself.

### EXAMPLE 2

In the following example, it is assumed that R=2.0 and that the magnitude of the complex signal vector S is 1.0. Such a situation is representative of a sick (i.e., low SpO₂ saturation) patient. As is illustrated in FIG. 3B, the slope of the complex signal vector S formed by combining the infrared and red signals S₁, S₂ has a slope of 2.0 and a length of 1.0. In this example, the projection of the complex signal vector S onto the infrared axis is now 0.447 and the projection of the complex signal vector S onto the red axis is now 0.894. Here, the red signal S₂ has a better S/N ratio than the infrared signal S₁. The complex signal vector S is rotated into the real axis by multiplying the complex signal vector S by the complex transformation vector:

$$\begin{matrix}T = {T}_{1} + i {T}_{2} \\ = \frac{1}{\sqrt{1 + {R}^{2}}} - i \frac{R}{\sqrt{1 + {R}^{2}}} \\ = \frac{1}{\sqrt{1 + {2.0}^{2}}} - i \frac{2.0}{\sqrt{1 + {2.0}^{2}}} \\ = 0.447 - 0.894 i\end{matrix}$$

The following result is obtained:

$$\begin{matrix}S * T = ( 0.447 + 0.894 i ) ( 0.447 - 0.894 i ) \\ = 0.1998 - 0.3996 i + 0.3996 i - 0.7992 {i}^{2} \\ = 1.0\end{matrix}$$

Here, the result obtained is over a 123% increase in signal strength as compared with using the infrared signal by itself.

Exemplary System For Implementing Plethysmographic Signal Processing Method

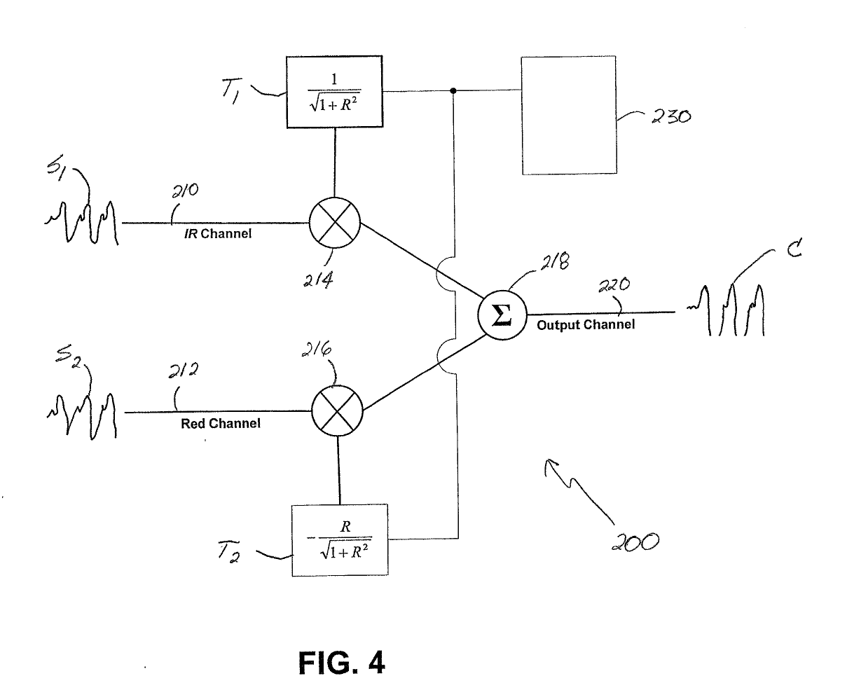

Referring now to FIG. 4, there is shown a block diagram of one embodiment of a system 200 for implementing the plethysmographic signal processing method of the present invention. In configuring the system 200, it has been recognized that the method of the present invention can be simplified. In this regard, assuming R is correct, it contains only noise and motion and therefore, only the real part of the result obtained when multiplying the complex signal vector S by the complex transformation vector T needs to be computed and the imaginary part of the result can be ignored.

The system 200 includes an infrared channel 210 for receiving an infrared plethysmographic signal S₁ thereon and a red channel 212 for receiving a red plethysmographic signal S₂ thereon. A first multiplier 214 takes as inputs the infrared signal S₁ received on the infrared channel 210 and a first multiplication factor T₁ and outputs the result of the first multiplication factor T₁ times the infrared signal S₁. A second multiplier 216 takes as inputs the red signal S₂ received on the red channel 212 and a second multiplication factor T₂ and outputs the result of the second multiplication factor T₂ times the red signal S₂. The results output by the first and second multipliers 214, 216 are directed to a summer 218 which adds the multiplication results together and outputs the composite signal C on an output channel 220 of the system 200.

The system 200 may be implemented in analog components, in which case the multiplication and summing operations are performed in continuous time. Alternatively, the system 200 may be implemented using digital technologies (e.g., in software executable by the processor 36 of the monitor unit of a pulse oximeter 10 such as described in connection with FIG. 1), in which case the multiplication and summing operations are performed on discrete time samples.

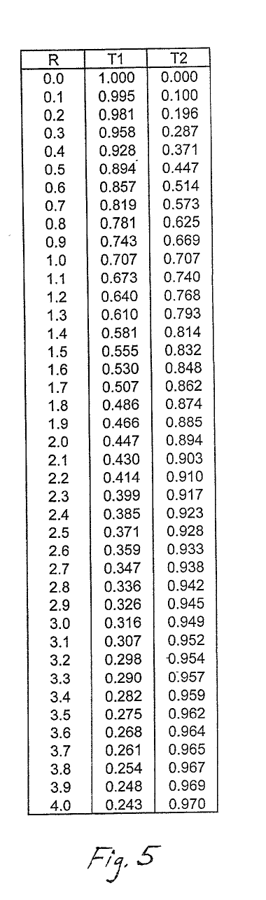

The first and second multiplication factors T₁, T₂ depend upon the R value and are computed in accordance with the previously described formulas. Since the R value typically changes infrequently, the first and second multiplication factors T₁, T₂ can be computed infrequently (e.g., only when the R value changes) to reduce the computational requirements of the system 200. Further computational efficiencies can be achieved by computing first and second multiplication factors T₁, T₂ corresponding with a range of incremental R values in advance and storing the pre-computed multiplication factors T₁, T₂ in a lookup table 230 accessible to the system 200 (e.g., on an EPROM chip). In this regard, first and second multiplication factors T₁, T₂ may be pre-computed for R values ranging, for example, from 0.40 to 1.40 in, for example, 0.01 increments (i.e. for R=0.98, 0.99, 1.00, 1.01, 1.02, . . . ). FIG. 5 shows an exemplary look-up table 230 wherein the R values are incremented from 0.0 to 4.0 in equal 0.1 increments. Numerous other R value ranges and increments, equal or unequal, may be utilized depending upon factors such as the amount of precision desired and the amount of memory available for storing the lookup table. When needed, the first and second multiplication factors T₁, T₂ corresponding with the current R value are read from the lookup table. If there are no entries in the lookup table for the current R value, interpolation techniques may be employed or the current R value may be appropriately rounded to obtain the first and second multiplication factors T₁, T₂.

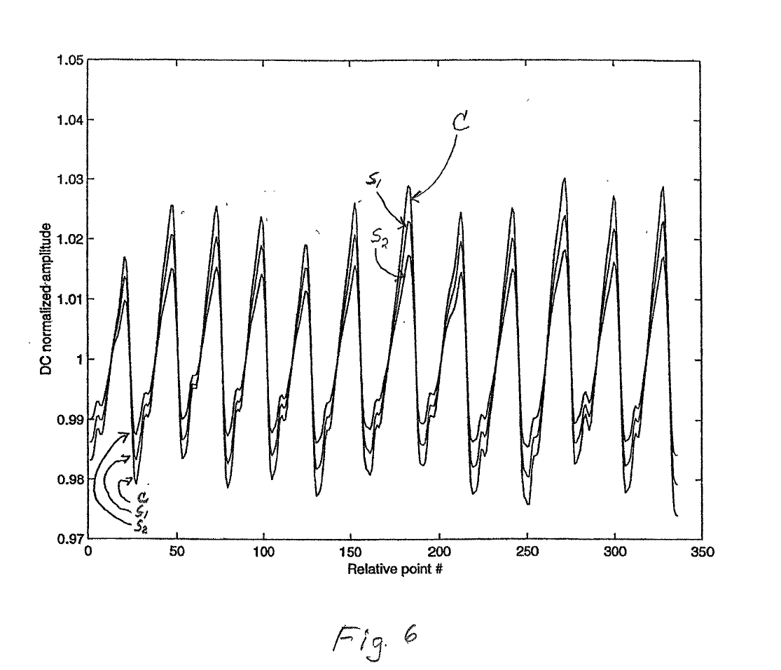

Plots of exemplary infrared plethysmographic and red plethysmographic signals S₁, S₂ and a composite signal C obtained using a system 200 such as described above implemented in computer software executable by a digital processor are shown in FIG. 6. In FIG. 6 the DC portions (i.e., the non-pulsatile components) of the signals S₁, S₂ and C have been normalized (i.e., set equal to 1.0) to emphasize the AC portions (i.e., the small pulsatile components) of the signals S₁, S₂ and C. As can be seen from FIG. 6, the composite signal C generated by the system 200 has a significantly greater peak-to-peak (i.e., high point to low point) amplitude difference than either the infrared or red plethysmographic signals S₁ and S₂ making it easier to perform heart-rate calculations and the like using the composite signal S and making the composite signal S easier to perceive visually on a display.

While various embodiments of the present invention have been described in detail, further modifications and adaptations of the invention may occur to those skilled in the art. However, it is to be expressly understood that such modifications and adaptations are within the spirit and scope of the present invention.

What is claimed is:

1. A signal processing method for use in plethysmography, said method comprising the steps of:
receiving at least two plethysmographic signals, each plethysmographic signal being associated with a particular wavelength;
multiplying each plethysmographic signal by an associated multiplication factor; and
generating a composite plethysmographic signal comprising a linear combination of the plethysmographic signals by adding the results of the multiplications.

2. The method of claim 1 wherein the multiplication factors are chosen to provide an improved S/N ratio of the composite signal as compared with S/N ratios of the plethysmographic signals over a specified range of SpO₂ levels.

3. The method of claim 2 wherein the specified range of SpO₂ levels is from 40% to 100%.

4 The method of claim 2 wherein the multiplication factors are dependent upon an R value that varies in accordance with an SpO₂ level in arterial blood circulated through a patient tissue site.

5. The method of claim 4 further comprising the step of:
obtaining the multiplication factors from a look-up table comprising sets of multiplication factors cross-referenced with corresponding incremental R values.

6. The method of claim 5 wherein the look-up table includes multiplication factors corresponding with incremental R values ranging from 0.4 to 1.4.

7. The method of claim 6 wherein the R values in the look-up table are incremented in equal increments, the increments being between 0.001 and 0.1.

8. The method of claim 4 wherein there are first and second plethysmographic signals and the wavelength associated with the first plethysmographic signal is between 800 and 950 nm and the wavelength associated with the second plethysmographic signal is between 600 and 700 nm.

9. The method of claim 8 wherein a first multiplication factor designated T₁ associated with the first plethysmographic signal is given by the following formula:
$${T}_{1} = \frac{1}{\sqrt{1 + {R}^{2}}}$$
and wherein a second multiplication factor designated T₂ associated with the second plethysmographic signal is given by the following formula:
$${T}_{2} = - \frac{R}{\sqrt{1 + {R}^{2}}}$$

10. The method of claim 9 wherein R is given by the following formula:
$$R = \frac{{dA}_{2}}{{dA}_{1}}$$
wherein dA₁ and dA₂ comprise differential absorption values obtained from the first and second plethysmographic signals, respectively.

11. The method of claim 1 wherein the plethysmographic signals comprise digital signals including pluralities of signal sample values taken at sequential temporal instances and said steps of multiplying and generating are performed for each temporally corresponding signal sample value.

12. A signal processing method for use in plethysmography, said method comprising the steps of:
receiving first and a second plethysmographic signals S₁ and S₂, the first and second plethysmographic signals S₁ and S₂ being associated with first and second wavelengths, respectively;
forming a complex signal vector S, wherein S is given by S=S₁+iS₂;
forming a complex transformation vector T from first and second scalar multiplication factors T₁ and T₂, wherein T is given by T=T₁+iTd₂; and
multiplying the complex signal vector S by the complex transformation vector T to generate a composite plethysmographic signal C.

13. The method of claim 12 wherein the scalar multiplication factors T₁ and T₂ are dependent upon an R value, wherein the R value varies in accordance with an SpO₂ level in arterial blood circulated through a patient tissue site.

14. The method of claim 13 further comprising the step of:
obtaining the scalar multiplication factors T₁ and T₂ from a look-up table comprising a plurality of pairs of scalar multiplication factors T₁ and T₂ cross-referenced with corresponding incremental R values.

15. The method of claim 14 wherein the look-up table includes multiplication factors T₁ and T₂ corresponding with incremental R values ranging from 0.4 to 1.4.

16. The method of claim 15 wherein the R values in the look-up table are incremented in equal increments, the increments being between 0.0001 and 0.1.

17. The method of claim 13 wherein the first scalar multiplication factor T₁ is given by the following formula:
$${T}_{1} = \frac{1}{\sqrt{1 + {R}^{2}}}$$
and wherein the second scalar multiplication factor T₁ is given by the following formula:
$${T}_{2} = - \frac{R}{\sqrt{1 + {R}^{2}}}$$

18. The method of claim 17 wherein R is given by the following formula:
$$R = \frac{{dA}_{2}}{{dA}_{1}}$$
wherein dA₁ and dA₂ comprise differential absorption values obtained from the first and second plethysmographic signals S₁ and S₂, respectively.

19. The method of claim 12 wherein the first and second plethysmographic signals S₁ and S₂ are digital signals including pluralities of signal sample values taken at sequantial temporal instances and said steps of multiplying and generating are performed for each temporally corresponding signal sample value.

20. The method of claim 12 wherein the first and second plethysmographic signals S₁ and S₂ are associated with infrared and red wavelengths, respectively.

21. A plethysmographic signal processing system comprising:
a first input channel for receiving a first plethysmographic signal thereon, said first plethysmographic signal being associated with a first wavelength;
a second input channel for receiving a second plethysmographic signal thereon, said second plethysmographic signal being associated with a second wavelength;
a first multiplier operable to receive the first plethysmographic signal and a first scalar multiplication factor as inputs and output a first product comprising the first plethysmographic signal multiplied by the first scalar multiplication factor;
a second multiplier operable to receive the second plethysmographic signal and a second scalar multiplication factor as inputs and output a second product comprising the second plethysmographic signal multiplied by the second scalar multiplication factor;
a summer operable to receive the first and second products as inputs and add the first and second products to output a composite signal comprising the sum of the first and second products.

22. The system of claim 21 wherein said first channel, said second channel, said first multiplier, said second multiplier, and said summer are implemented in software executable by a digital processor.

23. The system of claim 21 wherein said first channel, said second channel, said first multiplier, said second multiplier, and said summer comprise analog components.

24. The system of claim 21 wherein the first and second scalar multiplication factors are dependent upon an R value, wherein the R value varies in accordance with an SpO₂ level in arterial blood circulated through a patient tissue site.

25. The system of claim 24 further comprising:
a look-up table including a plurality of pairs of first and second scalar multiplication factors cross-referenced with corresponding incremental R values.

26. The system of claim 25 wherein said look-up table includes pairs of first and second multiplication factors corresponding with incremental R values ranging from 0.4 to 1.4.

27. The system of claim 26 wherein the R values in said look-up table are incremented in equal increments, said increments being between 0.001 and 0.1.

28. The system of claim 25 wherein the first scalar multiplication factor designated T₁ is given by the following formula:
$${T}_{1} = \frac{1}{\sqrt{1 + {R}^{2}}}$$
and wherein the second multiplication factor designated T₂ is given by the following formula:
$${T}_{2} = - \frac{R}{\sqrt{1 + {R}^{2}}}$$

29. The system of claim 28 wherein R is given by the following formula:
$$R = \frac{{dA}_{2}}{{dA}_{1}}$$
wherein dA₁ and dA₂ comprise differential absorption values obtained from with the first and second plethysmographic signals, respectively.

30. The system of claim 21 wherein the first and second plethysmographic signals are associated with infrared and red wavelengths, respectively.

31. The system of claim 21 further comprising:
at least one additional input channel for receiving at least one additional plethysmographic signal thereon, said at least one additional plethysmographic signal being associated with at least one additional wavelength; and
at least one additional multiplier operable to receive said at least one additional plethysmographic signal and at least one additional scalar multiplication factor as inputs and output at least one additional product comprising said at least one additional plethysmographic signal multiplied by said at least one additional scalar multiplication factor;
said summer being operable to receive the first, second, and at least one additional products as inputs and add the first, second, and at least one additional products together to output a composite signal comprising the sum of the first, second, and at least one additional products.
